(19) United States
(12) Patent Application Publication
(10) Pub. No.: US 2008/0255490 A1
(43) Pub. Date: Oct. 16, 2008

(54) Therapeutic foot appliance and method of use

(76) Inventor: Raija T. Daley, Hudson, OH (US)

Correspondence Address: RENNER KENNER GREIVE BOBAK TAYLOR & WEBER, FIRST NATIONAL TOWER FOURTH FLOOR, 106 S. MAIN STREET, AKRON, OH, 44308, US

(21) Appl. No.: 12/080,534

(22) Filed: Apr. 3, 2008

Related U.S. Application Data
(60) Provisional application No. 60/923,612, filed on Apr. 16, 2007.

(51) Int. Cl. A61F 5/00

(52) U.S. Cl. 602/30; 602/23

(57) ABSTRACT

A therapeutic device and method for its use are presented. The appliance and method are adapted to realign and straighten the toes of the foot in order to treat the effects of hammertoe, bunions, Morton's neuroma, and the like. Effectively, a harness is placed about the heel of the foot, the harness consisting of a foot strap and heel strap. Sleeves are placed about the particular toes of interest, being as few as one and as many as all of the toes on a foot. Elastic straps are interconnected between the toe sleeves and the foot strap in order to impart a force to the associated toe to straighten and/or realign the toe consistent with correcting a particular malady. The elastic strap may be connected and disconnected by means of hook and loop fasteners at the end of the strap and maintained upon the toe sleeves and foot strap. The positioning of the strap with respect to the toe sleeve and the foot strap effects the direction of the force applied to the toe in accordance with the malady to be treated.

### CROSS REFERENCE TO RELATED APPLICATION

This regular utility patent application claims the benefit of provisional patent application 60/923,612, filed Apr. 16, 2007.

### TECHNICAL FIELD

The invention herein resides in the art of therapeutic devices and, more particularly, to such devices for use in the treatment of foot ailments. More particularly, the invention relates to a therapeutic foot appliance that may be used during periods of rest to straighten and realign the toes and thereby treat problems and maladies associated with toes that otherwise are not straight or are misaligned.

### BACKGROUND ART

Foot ailments are among the most common types of human suffering. The foot is required to support and move significant amounts of weight upon a very small surface area, which necessarily requires flexibility and adaptability. Accordingly, foot ailments may evolve into significant problems and be attendant with great pain. Many of these problems are associated with toes that are either misaligned or have deviated from a straight orientation. Common among these problems associated with toe deviations are hammertoe, bunions, and Morton's neuroma. Each of these maladies can be traced to toes that have been forced out of alignment or have been allowed to move from alignment or from a straight orientation.

There is a need in the art for a therapeutic appliance or device that can serve to realign and re-straighten toes from their otherwise abnormal positions. This straightening and realigning has the tendency to abate the pain and minimize the impact of such anomalies. In the past, either surgery was required to correct deformation of the foot, or pads were employed to ease the pain. But, surgery was typically but partially remedial, and pads treated only the symptoms, not the problem. No effective non-surgical technique or appliance is known.

There is a need in the art for a simple and effective appliance that may be used in association with the toes of one's foot that can serve to straighten the toes or realign the toes and then correct the malady, not just mask the symptom.

### DISCLOSURE OF THE INVENTION

In light of the foregoing, it is a first aspect of the invention to provide a therapeutic foot appliance that may be effectively employed while the user is asleep.

Another aspect of the invention is the provision of a therapeutic foot appliance that is adaptable to attending to treating a variety of foot problems.

Still a further aspect of the invention is the provision of a therapeutic appliance that is adaptable to a wide variety of users, regardless of the size or shape of their feet.

Yet a further aspect of the invention is the provision of a therapeutic foot appliance that is particularly adapted for treating hammertoe, bunions, and Morton's neuroma.

A further aspect of the invention is the provision of a therapeutic foot appliance that can be worn without intrusion, provides relief without pain and is adapted for a wide variety of uses.

Still a further aspect of the invention is the provision of a method of using a therapeutic foot appliance that is adaptable to straightening and realigning toes in order to treat the maladies of hammertoe, bunions and Morton's neuroma.

Still a further aspect of the invention is the provision of a method of use of a therapeutic foot appliance that is simple and effective in implementation.

The foregoing and other aspects of the invention that will become apparent as the detailed description proceeds are achieved by a therapeutic foot appliance, comprising: at least one toe sleeve, each toe sleeve adapted for receiving a toe; a heel portion; and at least one elongated band for connecting each of said sleeves to the heel portion.

Other aspects of the invention that will become apparent herein are achieved by a therapeutic foot appliance, comprising: a foot strap; a heel strap secured to said foot strap; at least one toe sleeve for receiving a toe; and at least one elastic strap interconnected between said foot strap and said heel strap.

Yet further aspects of the invention are attained by a method for foot treatment, comprising: inserting certain toes of the foot into uniquely associated toe sleeves; attaching a foot strap and a heel strap to a heel portion of the foot; and elastically connecting said toe sleeves with one of said foot strap and heel strap, thereby applying a force to said certain of said toes toward the heel.

### DESCRIPTION OF DRAWINGS

For a complete understanding of the various aspects, structures and techniques of the invention, reference should be made to the following detailed description and accompanying drawings wherein:

FIG. 1 is a side perspective view of a foot appliance made in accordance with the invention;

FIG. 2 is a side elevational view of a foot receiving the foot appliance of FIG. 1;

FIG. 3 is a top view of a foot receiving the foot appliance of the invention;

FIG. 4 is a perspective view of a pair of toe sleeves, one open and one closed, having a reinforcing and/or cushioning member interposed therebetween;

FIG. 5 is an inside view of an elastic strap having hook and loop connectors at opposite ends thereof for use with the invention;

FIG. 6 is a side elevational view of a foot receiving the appliance of the invention for treatment of a bunion; and

FIG. 7 is a side elevational view of a foot (absent the large toe) showing its use in treatment of a hammertoe.

### BEST MODE FOR CARRYING OUT THE INVENTION

Referring now to the drawings and more particularly FIG. 1, it can be seen that a therapeutic foot appliance made in accordance with the invention is designated generally by the numeral 10. The foot appliance 10 includes a harness 12 that consists of a foot strap 14 with a substantially orthogonally connected heel strap 16. It will be appreciated with an understanding of the invention that the foot strap 14 and heel strap 16 may be made of any suitable material, such as elastic materials or inelastic materials such as leather or cloth.

Also included as a portion of the invention are a plurality of toe sleeves 18. It will be appreciated that as many as five (5) such toe sleeves might be included with the therapeutic foot appliance 10, there by shown in the illustration of FIG. 1. It will further be appreciated that the toe sleeves 18 may be either open ended or closed ended, depending upon the use to which they are to be put. In any event, it is most desired that the toe sleeves 18 be made of a soft material such as a thick cloth or the like. Alternatively, the sleeves 18 may be lined or padded with such a soft material.

Interposed between and interconnecting the toe sleeves 18 and the foot strap 14 are elastic straps 20, there being one provided in association with each toe sleeve 18. Typically, the straps 20 will be of a length slightly shorter than the distance between the connecting end of the toe sleeve 18 and the foot strap 14 when worn by a user. Accordingly, the elastic strap 20 extends and applies a pulling force on the sleeve 18 and associated toe, as will be appreciated below.

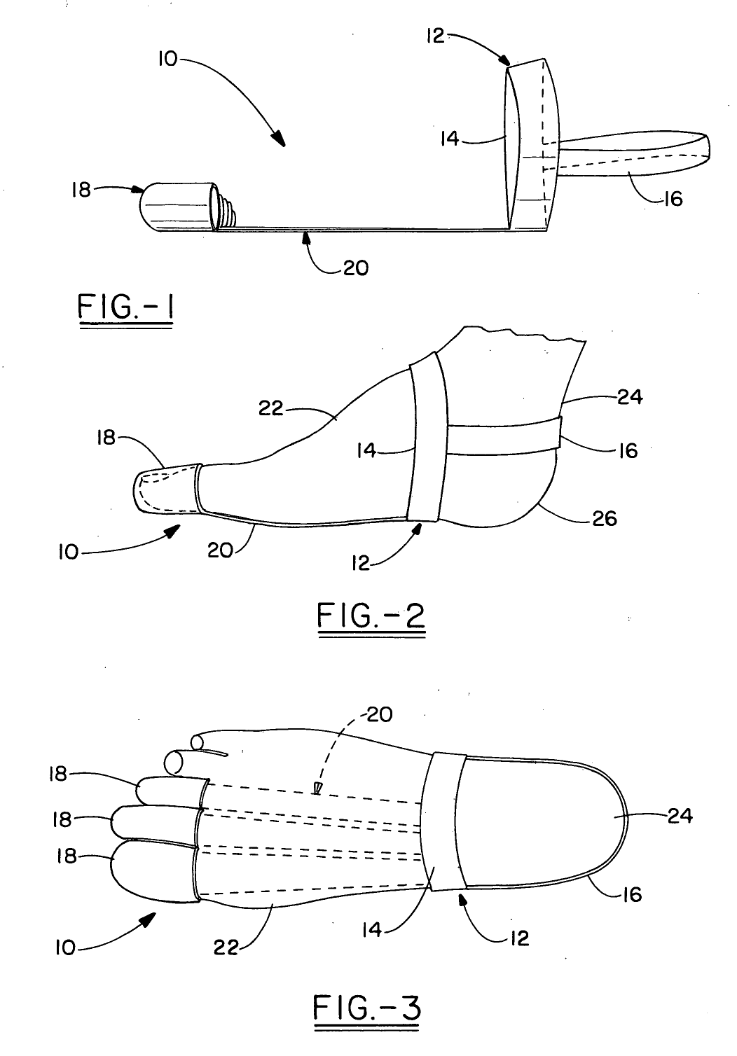

With reference now to FIG. 2, the therapeutic appliance 10 is shown as received upon a foot 22, with the foot strap 14 wrapping about the foot 22 and the heel strap 16 wrapped about the Achilles tendon area extending along the back of the ankle 24 above the heel 26. The toes of the foot are received within the sleeves 18, as shown.

With reference to FIG. 3, an illustrative top view of a foot 22 receiving the appliance 10 can be seen. Here, the elastic straps 20 are shown in phantom extending beneath the foot 22 and from respective ones of the toe sleeves 18 to the bottom of the foot strap 14. As will be appreciated below, in this position, the toes 18 are generally straightened and aligned, particularly for treatment of Morton's neuroma or the like.

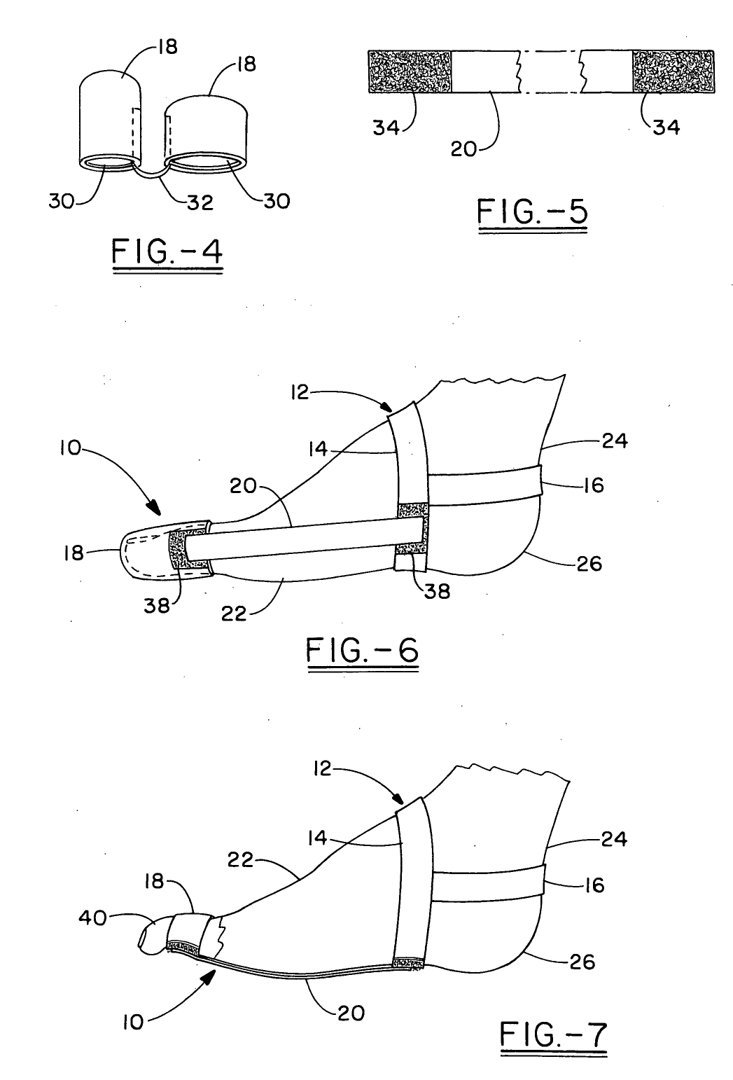

With reference now to FIG. 4, it can be seen that the invention contemplates closed end or open end sleeves 18, with the interior padded by means of an inner liner 30, inserts, or the like. Most preferably, the padding is part and parcel of the sleeves 18. The invention also contemplates that inserts may be received by and/or between the sleeves 18. The insert 32 may comprise a pad made of a soft cushioning material to protect the web of flesh between the toes. In this case, the pad 32 is horseshoe shaped, extending between adjacent sleeves 18. It is also contemplated that the insert pad may be of a rigid material in order to effect uniform force along the side of the toe for treatment of bunions or the like, as will be discussed below. In such a case, the insert may be placed into only one of the sleeves 18, and it will be substantially planar or slightly arcuate to conform to the side of the toe.

With reference now to FIG. 5, an appreciation can be obtained as to the structure of the straps 20. While the invention does contemplate that the elastic straps 20 may be fixedly secured at respective ends to the sleeves 18 and foot strap 14, it is preferred that the same be adjustable, both as to length and position. In this regard, it is contemplated that an extended end portion 34 of each of the straps 20 be provided with a hook and loop fastener or connector pad 38. It is contemplated that mating hook and loop materials be provided upon the sleeves 18 and foot strap 14 to accommodate the desired interconnection between the two. By providing substantial portions of the outer surface of the foot strap 14 with hook and loop material, and similarly providing a circumferential area of the sleeves 18 with such hook and loop material, the elastic strap 20 can be fastened or interconnected at almost any point between the two. It will be appreciated below that treatment for different maladies requires different positioning of the straps, accommodated by this feature.

With reference now to FIG. 6, an appreciation can be obtained of use of the appliance 10 for treatment of bunions. Those skilled in the art will appreciate that bunions occur as a consequence of the inward displacement of the large toe on a foot, causing the joint at the base of the toe to similarly displace and/or enlarge. The appliance 10 can be employed to bring the large toe outwardly, into its more natural position, and thereby eliminating the bunion effect. As shown in FIG. 6, the strap 20 of FIG. 5, having hook and loop fasteners 34 at the ends thereof, can be positioned alongside the foot between a sleeve 18 received upon the big toe and the foot strap 14. With the strap 20 stretched or sized sufficiently to ensure the imparting of force between the sleeve 18 and strap 14, the result is the effective outer movement of the large toe, to correct the effects of the bunion. It will, of course, be appreciated that the foot strap 14 is substantially immobile and fixed in place due to the retention of the heel strap 16.

A similar treatment is employed for hammertoe. This typically occurs with the toe immediately inward of the big toe on a foot. The toe has the tendency to arch at the first joint from its interconnection with the foot, as shown in FIG. 7. Treatment can be attained by attaching a properly sized and/or stretched elastic strap 20 between the hook and loop surfaces of the foot band 14 and toe sleeve 38 to impart a force therebetween. The straps 20 extend beneath the foot, as shown in FIGS. 3 and 7. This force tends to pull the first section of the toe adjacent the foot downwardly, thus straightening the hammertoe 40 and treating the effects thereof.

Those skilled in the art will appreciate that various maladies can be treated with the therapeutic foot appliance 10. The elastic straps 20 are readily removed and replaced. Their points of contact, or the sizes themselves, can dictate the amount of force they impart between an associated toe sleeve 18 and the foot band 14. The direction of force imparted to the toe is managed by the placement of the toe sleeve 18, the elastic strap 20, and the implementation of force distributing inserts 32, and the like. As with the bunion, the placement of force distributing inserts 32 within the sleeve 18 ensures that the force against the large toe pulling it outwardly is evenly distributed along the inner surface of the toe.

In use, it is preferred that the appliance 10 be employed when footwear is not being used, such as periods of relaxation or sleep. The size and strength of the elastic straps 20 is preferably such to impart a gentle continual force to the toe, without causing pain or further injury. It has been found that by using the appliance 5-8 hours a day, primarily during periods of sleep, corrective action on the toes of the foot have been realized sufficiently to significantly reduce the effects of bunions, hammertoe, and Morton's neuroma.

Thus it can be seen that the various aspects of the invention have been satisfied by the structure presented above. While in accordance with the patent statutes only the best mode and preferred embodiments of the invention have been presented and described in detail, the invention is not limited thereto or thereby. Accordingly, for an appreciation of the true scope and breadth of the invention reference should be made to the following claims.

What is claimed is:

1. A therapeutic foot appliance, comprising:
at least one toe sleeve, each toe sleeve adapted for receiving a toe;
a heel portion; and
at least one elongated band for connecting each of said sleeves to the heel portion.

2. The therapeutic foot appliance of claim 1, wherein said at least one toe sleeve has a bottom portion, and wherein the heel portion has a bottom portion on the underside of the foot wherein each of said bands connects the bottom portion of the toe sleeve with the bottom portion of the heel portion.

3. The therapeutic foot appliance of claim 2, wherein the elongated band comprises an elastic strap.

4. The therapeutic foot appliance of claim 3, wherein the heel portion comprises a foot band which fits around the foot and a heel band connected to the foot band and which goes around the Achilles tendon.

5. The therapeutic foot appliance of claim 3, wherein the elastic strap connects to the foot sleeve and heel portion by hook and loop fasteners.

6. The therapeutic foot appliance of claim 3, further comprising an interconnecting link which connects a first toe sleeve with a second immediately adjacent toe sleeve.

7. The therapeutic foot appliance of claim 6, wherein said interconnecting link comprises a padded cushion.

8. A therapeutic foot appliance, comprising:
a foot strap;
a heel strap secured to said foot strap;
at least one toe sleeve for receiving a toe; and
at least one elastic strap interconnected between said foot strap and said heel strap.

9. The therapeutic foot appliance according to claim 8, wherein said elastic strap is removably attachable to said foot strap and said toe sleeve.

10. The therapeutic foot appliance according to claim 9, wherein said elastic strap has hook and loop fasteners at each of opposite ends thereof.

11. The therapeutic foot appliance according to claim 8, wherein said at least one toe sleeve has a rigid reinforcement insert along at least one side thereof.

12. The therapeutic foot appliance according to claim 8, wherein said foot strap, heel strap and sleeve are formed of an elastic material.

13. The therapeutic foot appliance according to claim 8, wherein said at least one toe sleeve has a cushion interconnecting it with an adjacent toe sleeve.

14. A method for foot treatment, comprising:
inserting certain toes of the foot into uniquely associated toe sleeves;
attaching a foot strap and a heel strap to a heel portion of the foot; and
elastically connecting said toe sleeves with one of said foot strap and heel strap, thereby applying a force to said certain of said toes toward the heel.

15. The method for foot treatment according to claim 14, wherein said toe sleeve is elastically connected to said foot strap beneath the foot.

16. The method for foot treatment according to claim 15, wherein said interconnection of said toe sleeve and foot strap provides therapy for a hammertoe.

17. The method for foot treatment according to claim 15, wherein said interconnection of said toe sleeve and foot strap provides therapy for Morton's neuroma.

18. The method for foot treatment according to claim 14, wherein said sleeve is elastically connected to said foot strap alongside the foot.

19. The method for foot treatment according to claim 18, wherein said interconnection of said toe sleeve and joint strap provides therapy for a bunion.
